(19) United States
(12) Patent Application Publication
(10) Pub. No.: US 2007/0105234 A1
(43) Pub. Date: May 10, 2007

(54) Diagnosing equine hyperelastosis cutis

(75) Inventors: Cyprianna Ellen Swiderski, Starkville, MS (US); Ann Maura Rashmir-Raven, Starkville, MS (US); Marzia Pasquali, Salt Lake City, UT (US)

Correspondence Address: TRASK BRITT, P.O. BOX 2550, SALT LAKE CITY, UT, 84110, US

(73) Assignee: University of Utah Research Foundation, Salt Lake City, UT (US)

(21) Appl. No.: 11/584,710

(22) Filed: Oct. 20, 2006

Related U.S. Application Data
(60) Provisional application No. 60/728,575, filed on Oct. 20, 2005.

(51) Int. Cl. G01N 33/00

(52) U.S. Cl. 436/106

(57) ABSTRACT

A method of diagnosing equine hyperelastosis cutis which includes determining the ratio of deoxypyridinoline to pyridinoline of a subject horse and comparing the ratio of deoxypyridinoline to pyridinoline of the subject horse to a ratio of deoxypyridinoline to pyridinoline of a hyperelastosis cutis-free horse. The ratio of deoxypyridinoline to pyridinoline may be determined by a urine analysis using high performance liquid chromatography. In particular embodiments, a method includes determining the ratio of equine deoxypyridinoline to pyridinoline using antibodies configured to determine the ratio of equine deoxypyridinoline to pyridinoline. Also described is a method of diagnosing equine hyperelastosis cutis wherein the ratio of deoxypyridinoline to pyridinoline is determined by tissue analysis using high performance liquid chromatography.

### RELATED APPLICATIONS UNDER 35 U.S.C. § 119(e)

This application claims the benefit of U.S. Provisional Patent Application Serial No. 60/728,575, filed Oct. 24, 2005, for “DIAGNOSING EQUINE HYPERELASTOSIS CUTIS” the entire contents of which are hereby incorporated herein by this reference.

### FIELD OF THE INVENTION

This invention relates generally to medicine and veterinary diagnostics, and in particular, to a method for diagnosing equine hyperelastosis cutis or hereditary equine regional dermal asthenia.

### BACKGROUND

Hyperelastosis cutis (“HC”), also known as hereditary equine regional dermal asthenia (“HERDA”), is reported to be an autosomal recessive connective tissue disorder (see, Refs. 1 and 2). Horses affected with HC have extremely fragile thin skin that tears easily and exhibits impaired healing (see, Refs. 3-6). In horses with HC, the skin separates between the superficial and deep dermis, resulting in skin that is not securely attached in affected areas (see, Ref. 6). The condition may be found in Quarter Horse lineages tracing to the stallion “Poco Bueno” and his sire “King” (see, Ref. 1). A similar condition has been described in an Arabian cross-bred mare, a Thoroughbred gelding, a Hanoverian foal, and a Hafflinger horse.

Diagnosis of HERDA is based on the presence of typical cutaneous lesions in a young Quarter horse. Histopathology demonstrates thinning of the dermis, irregularities in the size, shape, and staining affinity of collagen fibers, reduced amounts of dermal collagen, and fragmentation and disorientation of collagen fibers. A characteristic lesion in which the upper and lower portions of the deep dermis separate, termed “zonal dermal separation,” has been described. A history of similar lesions in the sire, dam, siblings, or closely related horses is helpful diagnostically.

Hyperelastosis cutis is generally diagnosed after the horse is two years of age, as it enters training and clinical signs of the disease become evident. Unfortunately, by that time, significant, emotional and financial investments have been made. Because horses with HC cannot be ridden and are unsuitable for breeding due to the genetic nature of the disease, most are humanely destroyed.

Pedigree analysis indicates that the gene or genes that cause HC are inherited as autosomal recessive traits (see, Refs. 1 and 2). This means that in order for the offspring of an equine mating to be affected by HC, the progeny must inherit one copy of the defective gene from its sire and one copy from its dam. Therefore, both the sire and dam must have at least one copy of the recessive HC gene for the offspring to be affected. Horses that inherit two copies of the genetic defect become clinically affected, while horses that inherit a single copy of the defect from either their sire or dam are asymptomatic carriers of the genetic defect.

Identifying those individual horses that carry one or two copies of the HC defect within the Quarter Horse population is problematic because horses that have inherited only one copy of the HC defect are asymptomatic “carriers” of the trait, while horses that inherit two copies of the genetic defect may take years to demonstrate the condition. In either case, both carriers of a single copy of the HC defect (termed heterozygote or carriers) and those affected horses that have two copies of the defective HC gene (termed homozygotes) are sources for propagation of the genetic defect within the population. This situation is problematic because bloodlines known to carry the genetic defect causing HC are highly desirable in certain equestrian disciplines (see, Ref. 1). This increases the frequency of matings that produce both HC carrier and HC affected horses, thereby increasing the frequency of the genetic defect within the Quarter Horse population.

As far as is known, to date, researchers have not identified the genetic defect that causes HC. Only after the genetic defect responsible for HC is identified will it be possible to diagnose HC using testing procedures that identify the offending DNA abnormality in affected and carrier animals.

At this time no test exists that reliably identifies horses with HC. Currently, a diagnosis of HC relies upon a triad that includes pedigree analysis to identify high risk blood lines in the patient's lineage (see, Ref. 1), clinical signs consistent with HC such as patches of poorly attached “mushy” skin, skin sloughing and/or severe scarring (see, Refs. 3-5), as well as abnormal microscopic findings in skin biopsy (see, Refs. 6 and 7). Though microscopic examination of skin from horses with HC has shown some similar lesions, including a zone of separation in the deep dermis termed “zonal dermal separation” (see, Ref. 6), the distribution and severity of the lesions are variable and are not necessarily reliable for the diagnosis of HC (see, Ref. 7).

### SUMMARY OF THE INVENTION

Described is a method of diagnosing equine hyperelastosis cutis that comprises determining a ratio of deoxypyridinoline to pyridinoline of a subject horse, and comparing the ratio of deoxypyridinoline to pyridinoline of the subject horse to a ratio of deoxypyridinoline to pyridinoline of a hyperelastosis cutis-free horse, wherein the ratio of deoxypyridinoline to pyridinoline of the subject horse significantly exceeds that of the ratio of deoxypyridinoline to pyridinoline of a hyperelastosis cutis-free horse.

Additionally, described is a method of diagnosing equine hyperelastosis cutis, wherein the ratio of deoxypyridinoline to pyridinoline is determined by urine analysis, the method comprising using high performance liquid chromatography. Further described is a method of diagnosing equine hyperelastosis cutis wherein the ratio of deoxypyridinoline to pyridinoline is determined by urine analysis, the method comprising using antibodies configured to determine the ratio of equine deoxypyridinoline to pyridinoline. Also described is a method of diagnosing equine hyperelastosis cutis wherein the ratio of deoxypyridinoline to pyridinoline is determined by tissue analysis, the method comprising the use of high performance liquid chromatography.

In addition, also described is a method of diagnosing equine hyperelastosis cutis in which the ratio of deoxypyridinoline to pyridinoline in urine is greater than about 1.45 (e.g., greater than about 1.25).

### BRIEF DESCRIPTION OF THE DRAWINGS

FIG. 1 is a bar graph depicting the effect of furosemide (LASIX®) on urine DPD and PYD.

FIG. 2 is a bar graph depicting the effect of furosemide on urine DPD:PYD ratio.

FIG. 3 is a flow diagram illustrating a method of diagnosing HC.

### BEST MODE OF THE INVENTION

The described methods are significant for various reasons. The first is the method's ability to definitively identify a horse that is affected by HC (i.e., is homozygous for the HC trait) when the horse's lineage and/or clinical signs indicate that the horse could be homozygous for the HC trait. Second, the method has successfully identified and differentiated experimental offspring that are known to be homozygous for the HC trait (and therefore affected by HC) from horses that are known to be free from HC (because they are either heterozygous for HC or entirely lack the HC trait). Experimental animals bred to be free from HC were either heterozygous (derived from breedings of homozygous HC affected horses to horses from lineages known to be free from HC) or negative for the HC trait (derived from matings of horses from lineages known to be free from HC).

In the United States, HERDA is primarily seen in cutting horse lines and has an autosomal recessive mode of inheritance. As previously stated, a similar condition has been described in an Arabian cross-bred mare, a Thoroughbred gelding, a Hanoverian foal, and a Hafflinger horse. Skin lesions are generally noted following normally innocuous trauma such as pressure from the saddle. No sex predilection has been reported. Skin lesions may be solitary or multiple areas of loose, hyperextensible skin that tears easily, and exhibits impaired healing with extensive dermal scarring. In severely affected areas, the dermis may be completely separated from the underlying tissue. The most common location is the dorsal body surface, although the lesions have also been described in the thoracic region, shoulder areas, and limbs. Affected areas may develop subcutaneous hematomas and pain may be elicited when traction is applied to the edge of the defect. Aside from minimizing trauma and avoiding excessive exposure to sunlight, there is no treatment. Affected horses, as well as sires and dams of affected horses, should be removed from breeding programs.

Due to the autosomal recessive nature of the condition, foals from HC affected mares and HC affected stallions may be diagnosed as HC affected, using a method described herein, even though they have not had sufficient time for the classic lesions of HC to manifest. By virtue of its ability to identify HC affected horses prior to the onset of clinical signs, essentially independent of age, this diagnostic method is valuable to owners who typically have several years of time and finances invested in these horses prior to recognizing HC and the fact that the animal can serve no useful purpose. From a humane standpoint, HC horses can be identified and removed from both the training and breeding population, thereby minimizing their pain and suffering.

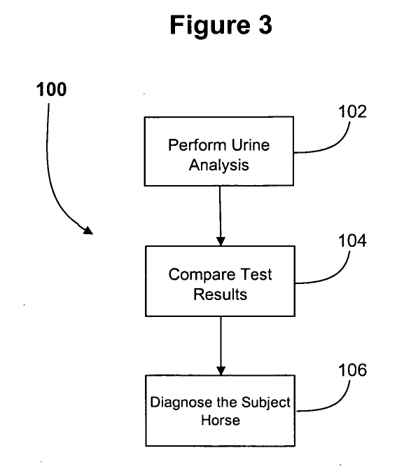

Referring now to FIG. 3, which depicts a method of diagnosing HC, diagnostic method 100, according to a preferred embodiment of the invention, involves three major steps. The three major steps are perform urine analysis 102, compare test results 104, and diagnose the subject horse 106.

Urine analysis 102 includes collecting a urine sample, measuring the deoxypyridinoline and pyridinoline levels in the urine, and determining the ratio of deoxypyridinoline to pyridinoline (DPD to PYD) in the urine. The preferred analysis method used to measure deoxypyridinoline and pyridinoline levels and determine the ratio of DPD to PYD in the urine is high performance liquid chromatography (HPLC).

However, under appropriate circumstances, other analysis methods may be used to quantify deoxypyridinoline and pyridinoline levels, including mass spectrometry and various chromatographic variations or techniques. Both deoxypyridinoline and pyridinoline are excreted in urine as products of collagen degradation, in peptide-bound and free forms.

First, the urine samples are hydrolyzed to release the peptide-bound pool. The cross-links, deoxypyridinoline and pyridinoline, are then extracted from the hydrolysate by fractionation on a cellulose column and analyzed by HPLC. The results of this analysis are reported as a ratio of DPD to PYD. As is understood by those skilled in the art, under appropriate circumstances, elements of performing the HPLC may vary, such as sample preparation, column type and size, and elution conditions, depending on the analysis sample (see, Ref. 27, the contents of which are incorporated by this reference).

Once the ratio of DPD to PYD in the urine is determined, the test results 104 are compared, as shown in FIG. 3. This is accomplished by comparing the urine analysis 102 DPD to PYD ratio of the subject horse to ratios of DPD to PYD in the urine of horses that are known to lack the HC trait. In certain embodiments, analysis of the DPD to PYD ratios in the urine of horses affected with HC (i.e., are homozygous for the HC trait), indicate that a randomly selected affected animal would be expected to have a DPD to PYD ratio in excess of 1.45, with 99% confidence. In contrast, a randomly selected control animal that either lacks, or is heterozygous for the HC trait is expected to have a DPD to PYD ratio less than 0.47, again with 99% confidence.

After the comparison of test results 104, it is then possible to diagnose the subject horse 106. In addition to the statistically significant increased DPD to PYD ratio, it may also be preferable to review the clinical symptoms, if any, of the subject horse as well the subject horse's lineage. Horses descended from “Poco Bueno” and “King P-234” and some Arabian-cross horses are especially suitable for analysis.

Other embodiments of the present invention include determining the DPD to PYD ratio in tissues, such as, but not limited to, skin or cultured skin fibroblasts, of the subject horse. If the subject horse is affected with HC, the ratio of DPD to PYD in the tissue is expected to be significantly higher than the DPD to PYD ratio determined in the same tissue derived from a horse that does not have HC.

Another embodiment of the invention includes measuring the DPD to PYD ratios in equine urine using an antibody-based method similar to that available for human specimens. Kits currently available to measure pyridinoline and deoxypyridinoline in human urine specimens include Metra PYD EIA kit and Metra DPD EIA kit, manufactured by Quidel. Antibodies, antibody fragments, aptamers or the like (collectively “antibodies”) configured to bind to equine forms of deoxypyridinoline and pyridinoline may be used to measure the relative amount of each moiety in a specimen using traditional means such as ELISA, RIA, immunoprecipitation, dipstick, or the like. Therefore, another embodiment of the present invention includes kits, including these antibodies, for measuring the DPD to PYD ratios in equine urine samples.

In certain embodiments, the invention also includes a kit for diagnosing equine hyperelastosis cutis in a horse, said kit comprising: means for determining a concentration of deoxypyridinoline in a sample taken from the horse, and means for determining a concentration of pyridinoline in the sample. Such means include high performance liquid chromatography or antibody analysis of a urine or other sample taken from the horse under consideration.

The method is also significant because it links HC to a very specific set of genetically encoded biochemical defects that cause defective hydroxylation of collagen in humans. Defective hydroxylation decreases the tensile strength of collagen.

Humans with urine deoxypyridinoline to pyridinoline ratios in the range identified in HC affected horses have disease manifestations quite similar to horses with HC. The human genetic mutations that increase the urine deoxypyridinoline to pyridinoline (DPD to PYD) ratio to the magnitude observed in horses with HC result almost exclusively from homozygous or compound heterozygous mutations in the allele(s) that code for isomers of the enzyme PLOD (a.k.a. lysl-hydroxylase), particularly PLOD1 (see, Refs. 8 and 9). This third finding not only refines the set of genes to be examined to identify the genetic defect responsible for HC and develop a genetic test, but also has implications for HC as a naturally occurring model of human disease. More specifically, the magnitude of elevation of the DPD to PYD ratio in the urine of HC horses specifically mirrors findings in the urine of humans with Ehlers-Danlos Syndrome VI (EDS VI), kyphoscoliotic form (see, Refs. 10-12).

The invention in certain embodiments thus includes an improvement in a method of assisting the diagnosis, treatment and/or prevention of Ehlers-Danlos Syndrome in humans, the improvement comprising using a horse suffering from equine hyperelastosis cutis as an animal model for said diagnosis, treatment, and/or prevention. For example, the horse suffering from equine hyperelastosis cutis may be used as a pharmacological animal model to test the efficacy of biologically active agents on symptoms associated with Ehlers-Danlos Syndrome and equine hyperelastosis cutis. The horse suffering from equine hyperelastosis cutis may also be used as a pharmacological animal model to test the efficacy of biologically active agents in preventing symptoms associated with Ehlers-Danlos Syndrome and equine hyperelastosis cutis.

EDS VI is well recognized to result from a collection of mutations in the DNA that codes for enzymes that hydroxylate lysine residues in collagen (see, Refs. 8 and 13-16). Collagen hydroxylation is critical to the formation of pyridinium cross-links responsible for the tensile strength of collagen (see, Refs. 17-19) and the correlation between decreased hydroxylation of collagen pyridinium cross-links in the urine of HC horses and enzymatic defects capable of causing HC is significant. Manifestations of EDS VI in human patients include hyperextensible and friable skin, joint hypermobility, and, less commonly, scoliosis, ocular fragility, and arterial rupture (see, Refs. 16, 20 and 21).

EDS VI results from the defective hydroxylation of lysl residues on procollagen peptides (see, Refs. 19 and 22-24). EDS VI patients, therefore, have decreased hydroxylysine residues that are essential for forming covalent linkages (see, Ref. 24), specifically pyridinoline and deoxypyridinoline in mature collagen molecules (see, Refs. 23 and 25). Pyridinoline, the predominant pyridinium cross-link found in normal patients, results from three hydroxylysine residues (see, Ref. 26). In contrast, deoxypyridinoline requires only two hydroxylysine residues and one lysl residue (see, Ref. 26). When compared to normal patients, EDS VI patients have reduced total pyridinium cross-links in skin, cultured skin fibroblasts, and urine with deoxypyridinoline levels far exceeding pyridinoline (see, Refs. 11 and 25). Consequently, the urinary excretion of deoxypyridinoline, relative to pyridinoline is markedly increased. This is exploited diagnostically in EDS VI patients whose urine deoxypyridinoline to pyridinoline (DPD to PYD) ratios exceed 2.0.

As is more thoroughly described herein, the effect of furosemide administration on the ratio of DPD:PYD in urine was also determined. Urine was aseptically collected from four horses with HERDA and six control horses via urethral catheterization. Following evacuation of the bladder, furosethide (0.5 mg/kg IV) was administered and a voided urine sample was collected 15 minutes following the administration of furosemide. Despite dilution of the urine, as supported by a significant decrease in the urine creatinine and specific gravity, there was not a statistically significant change in the DPD:PYD ratio following furosemide injection. Furosemide administration does not significantly alter the DPD:PYD ratio.

The invention is further explained with the aid of the following illustrative Examples.

### EXAMPLES

### Example I

The Ratio Of Urine Deoxypyridinoline To Pyridinoline Identifies Horses With Hyperelastosis Cutis

Elevated urine ratios of the collagen pyridinium cross-links deoxypyridinoline (DPD) and pyridinoline (PYD) were used to diagnose EDS Type VIA (collagen lysyl-hydroxylase deficiency).

A blinded comparison of DPD:PYD ratios was conducted in the urine of 39 clinically normal horses and 19 horses previously diagnosed with HERDA. Total DPD and PYD in urine were determined by high pressure liquid chromatography. The ratio of DPD:PYD was significantly higher in horses known to have HERDA than in healthy controls (p<0.0001) using a two-tailed independent samples t test for unequal variances. The mean ratio difference between groups was 2.48, with a 95% confidence interval of (2.30, 2.67).

Pyridinium cross-links are the intermolecular bonds of collagen. DPD and PYD are derived from two and three hydroxylysine residues, respectively, with PYD being the predominant Type I collagen crosslink in normal individuals. These data suggest that abnormal hydroxylation of collagen lysine residues is significant in the pathogenesis of HERDA.

### Example II

Example I demonstrates that horses with HERDA can be identified by a statistically significant increase in the ratio of deoxypyridinoline (DPD) to pyridinoline (PYD) in their urine. Collagen lysyl hydroxylase hydroxylates lysine residues on procollagen peptides. Decreased availability of hydroxylysine residues limits the formation of the covalent pyridinium crosslinks, pyridinoline (Pyr) and deoxypyridinoline (Dpyr), in mature collagen molecules. Pyridinoline, the predominant pyridinium crosslink found in normal subjects, results from three hydroxylysine residues. In contrast, deoxypyridinoline requires only two hydroxylysine residues and one lysl residue. In this Example, we determine the effect of administration of the diuretic furosemide on the DPD:PYD ratio in equine urine.

### MATERIALS AND METHODS

Horses—Eleven adult horses (3 to 17 years old) were used to study the effect of furosemide administration on the ratio of DPD:PYD in the urine. Urine was obtained from five Quarter horses (two mares, one stallion, and one gelding) that had been previously diagnosed with hyperelastosis cutis on the basis of characteristic skin lesions, at risk pedigree, and an elevated DPD:PYD ratio in the urine. Control urine samples were obtained from four healthy Quarter horses (three mares and one stallion) with normal urine DPD:PYD ratios and lacking characteristic skin lesions. Urine samples were also obtained from two healthy non-Quarter horses (one Appaloosa mare and one Thoroughbred gelding). Horses were evaluated clinically before and after sample collection. All horses had normal vital parameters based on physical examination. Weight was estimated using a Weigh-Tape to measure the horse's heart girth. The experiment protocol for this project was approved by the Institutional Animal Care and Use Committee at Mississippi State University.

Sample collection—Horses were restrained in stocks and a physical examination was performed. The vulva or penis was aseptically cleansed with a BETADINE® solution. The urethras of the mares were catheterized with digital assistance using either a sterile plastic pipette or a curved metal catheter. The urethras of the males were catheterized with a stallion catheter under tranquilization. A pre-drug urine sample was collected, then the urinary bladder was completely voided. Initially, the urinary catheters were maintained indwelling in a subset of horses until the end of the experiment. Palpation of the urinary bladder per rectum was performed to confirm that the bladder was successfully voided. Furosemide (SALIX®′a8-Intervet, Inc., Millsboro, Del.) at 0.05 mg/kg was administered intravenously and first post-furosemide administration urine sample was collected via either free catch or via the urinary catheter. Horses were housed in stalls and provided with grass hay during the collection of the post-furosemide samples.

Sample Analysis—DPD and PYD were quantified using reverse phase HPLC at ARUP Laboratories, Salt Lake City, Utah. Samples were maintained at −80° C. until analysis. DPD and PYD are excreted in the urine, both in free and peptide-bound forms, as a result of collagen degradation. To release the peptide-bound cross-links, the urine samples were hydrolyzed in 6 M hydrochloric acid, under vacuum, for 16 hours at 150° C. Pyridinium cross-links were then extracted from the hydrolysate by fractionation through a cellulose column, according to established procedures. The eluates containing DPD and PYD were lyophilized, reconstituted in 1% heptafluorobutyric acid (HFBA), and analyzed by reverse-phase HPLC, using a Waters 2695 Alliance HPLC System, equipped with a fluorescence detector (Waters 2475; Emission=297 nm, Excitation=395 mn) and controlled by a computerized unit (Empower Software). The reverse phase column used for the separation of pyridinium cross-links was a Waters Nova-Pak C18 column (4 μ′b5 m; 15 cm×3.9 mm). Eluant A was 0.18% n-heptafluorobutyric acid (HFBA) in 12% acetonitrile. Eluant B was 0.18% HFBA in 100% acetonitrile. The column was equilibrated in 95% A and 5% B. The samples were eluted in 15 minutes with the same isocratic solvent composition at a flow rate of 0.7 ml/minute. Pyridinium cross-link concentration was calculated using a four-levels calibration curve obtained with an external standard. Standards for Dpd and Pyd were purchased from Quidel Corporation, San Diego, Calif. Urinary pyridinium cross-link concentration was normalized to urinary creatinine, measured by a Beckman Creatinine Analyzer 2. Intra-assay variability was 2.8% and 2.6% for Pyd and Dpd; inter-assay variability was 4.0% and 5.0%.

### RESULTS

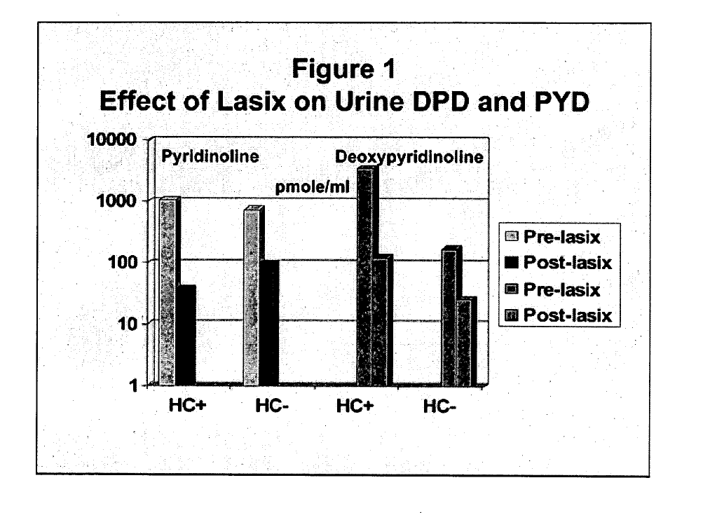

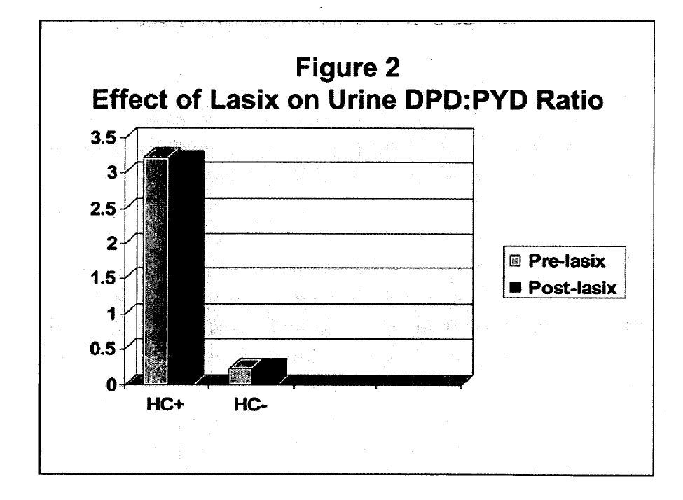

Our data indicate that the administration of furosemide is associated with a statistically significant decrease in the concentration of urinary pyridinium byproducts, deoxypyridinoline and pyridinoline in the urine of HC affected and non-affected animals (FIG. 1). When DPD and PYD concentrations were normalized to the urine creatinine concentration, there was no statistical difference in the concentrations of DPD and PYD associated with furosemide administration (data not shown). Significantly, furosemide administration did not alter the ratio of urine DYP:PYD in HC affected nor control animals, when normalized for urinary creatinine (FIG. 2).

Furosemide decreased the concentrations of DPD and PYD in the urine of horses with and without hyperelastosis cutis. However, normalization of the DPD and PYD concentrations to creatinine corrected the dilution effect of furosemide. This Example illustrates that furosemide administration had no significant effect on the ratio of deoxypyridinoline to pyridinoline in the urine of horses with hyperelastosis cutis. Accordingly, urine collected following the administration of furosemide is suitable as a diagnostic sample for HC using the DPD:PYD ratio.

Although the applicants have described applicants' preferred embodiments of this invention, it will be understood that the broadest scope of this invention, including referenced documents, includes such modifications as additional or different interactions and materials. Further, many other advantages of applicants' invention will be apparent to those skilled in the art from the above descriptions, incorporated referenced documents, and the below claims.

### REFERENCES

(The contents of the entirety of each of which are hereby incorporated by this reference).

1. Rashmir-Raven A. M., N. J. Winand, R. W. Read, R. M. Hopper, P. L. Ryan, M. H. Poole and H. M. Erb. Equine Hyperelastosis Cutis Update, AAEP Proceedings, 47-50 (2004).

2. Tryon R. C., S. D. White, T. R. Famula, P. C. Schultheiss, D. W. Hamar and D. L. Bannasch. Inheritance of Hereditary Equine Regional Dermal Asthenia in Quarter Horses, Am. J. Vet. Res. 66:437-442 (2005).

3. Lerner D. J. and M. D. McCracken. Hyperelastosis in Two Horses, Journal of Equine Medicine and Surgery, 2:350-352 (1978).

4. Bridges C. H. and W. C. McMullan. Dermatosporaxis in Quarter Horses, Proceedings of the American College of Veterinary Pathologists, Toronto, Ontario: ACVP, 22 (1984).

5. Hardy M. H., K. R. Fisher, O. E. Vrablic, et al. An Inherited Connective Tissue Disease in the Horse, Laboratory Investigation 59:253-262 (1988).

6. Brounts S. H., A. M. Rashmir-Raven, and S. S. Black. Zonal Dermal Separation: A Distinctive Histopathological Lesion Associated with Hyperelastosis Cutis in a Quarter Horse, Vet. Dermatol. 12:219-224 (2001).

7. White S. D., V. K. Affolter, D. L. Bannasch, P. C. Schultheiss, et al. Hereditary Equine Regional Dermal Asthenia (“Hyperelastosis Cutis”) in 50 Horses: Clinical, Histological, Immunohistochemical, and Ultrastructural Findings, Vet. Dermatol. 15:207-217 (2004).

8. Yeowell H. N. and L. C. Walker. Mutations in the Lysyl Hydroxylase I Gene that Result in Enzyme Deficiency and the Clinical Phenotype of Ehlers-Danlos Syndrome Type VI, Mol. Genet. Metab. 71(1-2):212-24 (September-October 2000).

9. Walker L. C., A. S. Teebi, J. C. Marini, A. De Paepe, F. Malfait, P. Atsawasuwan, M. Yamauchi and H. N. Yeowell. Decreased Expression of Lysyl Hydroxylase 2 (LH2) in Skin Fibroblasts from Three Ehlers-Danlos Patients does not Result from Mutations in Either the Coding or Proximal Promoter Region of the LH2 Gene, Mol. Genet. Metab. 83:312-321 (2004).

10. Pasquali M., P. P. Dembure, M. J. Still and L. J. Elsas. Urinary Pyridinium Cross-links: A Noninvasive Diagnostic Test for Ehlers-Danlos Syndrome Type VI, New Eng. J. Med. 331:132-3, 1994.

11. Pasquali M., M. J. Still, P. P. Dembure and L. J. Elsas. Pyridinium Cross-links in Heritable Disorders of Collagen, Am. J. Hum. Genet. 57:1508-10 (1995).

12. Steinmann B., D. R. Eyre and P. Shao. Urinary Pyridinoline Cross-links in Ehlers-Danlos Syndrome Type VI, Am. J. Human Genet. 57:1505-1508 (1995).

13. Pousi B., T. Hautala, J. C. Hyland, J. Schroter, B. Eckes, K. I. Kivirikko and R. Myllyla. A Compound Heterozygote Patient with Ehlers-Danlos Syndrome Type VI has a Deletion in one Allele and a Splicing Defect in the Other Allele of the Lysyl Hydroxylase Gene, Hum. Mutation 11(1):55-61 (1998).

14. Brinckmann J., Y. Acil, S. Feshchenko, E. Katzer, R. Brenner, A. Kulozik and S. Kugler. Ehlers-Danlos Syndrome Type VI: Lysyl Hydroxylase Deficiency due to a Novel Point Mutation (W612C), Arch. Dermatol. Res. 290(4):181-6 (April 1998).

15. Heikkinen J., B. Pousi, M. Pope and R. A. Myllyla. Null-mutated Lysyl Hydroxylase Gene in a Compound Heterozygote British Patient with Ehlers-Danlos Syndrome Type VI, Hum. Mutat. 14(4):351 (October 1999).

16. Yeowell H. N. and S. R. Pinnell. The Ehlers-Danlos Syndromes, Seminars in Dermatology 12:229-240 (1993).

17. Davis N. R., O. M. Risen and G. A. Pringle. Stable, Nonreducible Cross-links of Mature Collagen, Biochemistry 14:2031-2036 (1975).

18. Oxlund H., M. Barckman, G. Ortoft and T. T. Andreassen. Reduced Concentrations of Collagen Cross-links are Associated with Reduced Strength of Bone, Bone 17:365S-371S (1995).

19. Uitto J. and J. R. Lichtenstein. Defects in the Biochemistry of Collagen in Diseases of Connective Tissue, Journal of Investigative Dermatology 66:59-79 (1976).

20. Wenstrup R. J., S. Murad and S. R. Pinnell. Ehlers-Danlos Syndrome Type VI: Clinical Manifestations of Collagen Lysyl Hydroxylase Deficiency, Journal of Pediatrics 115:405-409 (1989).

21. Beighton P., A. De Paepe, B. Steinmann, P. Tsipouras and R. J. Wenstrup. Ehlers-Danlos Syndromes: Revised Nosology, Villefrache, 1997, Am. J. Med. Genet. 77:31-37 (1998).

22. Eyre D., P. Shao, M. A. Weis and B. Steinmann. The Kyphoscoliotic Type of Ehlers-Danlos Syndrome (Type VI): Differential Effects on the Hydroxylation of Lysine in Collagens I and II Revealed by Analysis of Cross-linked Telopeptides from Urine, Molecular Genetics and Metabolism 76:211-216 (2002).

23. Uzawa K., H.N. Yeowell, K. Yamamoto, Y. Mochida, H. Tanzawa and M. Yamauchi. Lysine Hydroxylation of Collagen in a Fibroblast Cell Culture System, Biochemical and Biophysical Research Communications 305:484-487 (2003).

24. Tajima S., S. Murad and S. R. Pinnell. A Comparison of Lysyl Hydroxylation in Various Types of Collagen from Type VI Ehlers-Danlos Syndrome Fibroblasts, Collagen and Related Research 3:511-515 (1983).

25. Pasquali M., M. J. Still, T. Vales, R. I. Rosen, J. D. Evinger, P. P. Dembure, et al., Abnormal Formation of Collagen Cross-links in Skin Fibroblasts Cultured from Patients with Ehlers-Danlos Syndrome Type VI, Proc. Assoc. Am. Physicians 109:33-41 (1997).

26. Eyre D. R., M. A. Paz and P. M. Gallop. Cross-linking in Collagen and Elastin, Ann. Rev. Biochem. 53: 717-748 (1984).

27. Uebelhart D., E. Gineyts, M. C. Chapuy and P. D. Delmas. Urinary Excretion of Pyridinium Crosslinks: A new Marker of Bone Resorption in Metabolic Bone Disease, Bone Miner. 8:87-96 (1990).

28. Borges A. S., L. G. Conceicao, A. L. G. Alves, et al. Hereditary equine regional dermal asthenia in three related Quarter horses in Brazil. Veterinary Dermatology 2005; 16:125-130.

29. Stannard A. A. Stannard's illustrated equine dermatology notes. Congenital diseases: epitheliogenesis imperfecta, epitheliogenesis bullosa and hyperelastosis cutis. Veterinary Dermatology 2000; 11:211-215.

30. Graves M. R., A. M. Rashmir-Raven, R. L. Linford, et al. Clinical Snapshot: Hyperelastosis Cutis case presentation. Compendium September 2001; 827, 837.

31. Elsas L. J., R. L. Miller, and S. R. Pinnell. Inherited human collagen lysyl hydroxylase deficiency: Ascorbic acid response. J. Pediatr. 1978; 92:378-384.

32. Swiderski C. E., M. Pasquali, L. Schwarz, et al. The Ratio of Urine Deoxypyridinoline to Pyridinoline Identifies Horses with HyperElastosis Cutis (a.ka. Hereditary Equine Regional Dermal Aesthenia, HERDA). JVIM 2006; 20:802.

What is claimed is:

1. A method of diagnosing equine hyperelastosis cutis, said method comprising:
determining a ratio of deoxypyridinoline to pyridinoline of a subject horse; and
comparing the ratio of deoxypyridinoline to pyridinoline of the subject horse to a ratio of deoxypyridinoline to pyridinoline of a hyperelastosis cutis-free horse,
wherein the ratio of deoxypyridinoline to pyridinoline of the subject horse significantly exceeds that of the ratio of deoxypyridinoline to pyridinoline of a hyperelastosis cutis-free horse.

2. The method according to claim 1, wherein the ratio of deoxypyridinoline to pyridinoline is determined by urine analysis comprising the use of high performance liquid chromatography.

3. The method according to claim 1, wherein the ratio of deoxypyridinoline to pyridinoline is determined by analyzing the urine of the subject horse with antibodies so as to determine the ratio of equine deoxypyridinoline to pyridinoline.

4. The method according to claim 1, wherein the ratio of deoxypyridinoline to pyridinoline is determined by tissue analysis comprising the use of high performance liquid chromatography.

5. The method according to claim 1, wherein the ratio of deoxypyridinoline to pyridinoline in urine is greater than 1.25.

6. The method according to claim 5, wherein the ratio of deoxypyridinoline to pyridinoline in urine is greater than 1.45.

7. The method according to claim 1, wherein the method of diagnosing further comprises a review of the lineage of the subject horse.

8. A method of diagnosing equine hyperelastosis cutis in a horse of quarter horse lineage, said method comprising:
determining a ratio of deoxypyridinoline to pyridinoline of the horse of quarter horse lineage; and
comparing the ratio of deoxypyridinoline to pyridinoline of the horse to a ratio of deoxypyridinoline to pyridinoline of a hyperelastosis cutis-free horse,
wherein the ratio of deoxypyridinoline to pyridinoline of the subject horse significantly exceeds that of the ratio of deoxypyridinoline to pyridinoline of a hyperelastosis cutis-free horse.

9. The method according to claim 8, wherein the ratio of deoxypyridinoline to pyridinoline in urine indicative of equine hyperelastosis cutis is greater than about 1.25.

10. The method according to claim 9, wherein the ratio of deoxypyridinoline to pyridinoline in urine indicative of equine hyperelastosis cutis is greater than about 1.45.

11. An improvement in a method of assisting the diagnosis, treatment and/or prevention of Ehlers-Danlos Syndrome in humans, the improvement comprising:
using a horse suffering from equine hyperelastosis cutis as an animal model for said diagnosis, treatment, and/or prevention.

12. The improvement of claim 11 wherein the horse suffering from equine hyperelastosis cutis is used as a pharmacological animal model to test the efficacy of biologically active agents on symptoms associated with Ehlers-Danlos Syndrome and equine hyperelastosis cutis.

13. The improvement of claim 11 wherein the horse suffering from equine hyperelastosis cutis is used as a pharmacological animal model to test the efficacy of biologically active agents in preventing symptoms associated with Ehlers-Danlos Syndrome and equine hyperelastosis cutis.

14. A kit for diagnosing equine hyperelastosis cutis in a horse, said kit comprising:
means for determining a concentration of deoxypyridinoline in a sample taken from the horse, and
means for determining a concentration of pyridinoline in the sample.

15. The kit according to claim 14, wherein the means for determining a concentration of deoxypyridinoline in the sample comprises high performance liquid chromatography.

16. The kit according to claim 14, wherein the means for determining a concentration of pyridinoline in the sample comprises high performance liquid chromatography.

17. The kit according to claim 14, wherein the means for determining a concentration of deoxypyridinoline in the sample comprises antibodies.

18. The kit according to claim 14, wherein the means for determining a concentration of pyridinoline in the sample comprises antibodies.

19. The kit according to claim 16, wherein the means for determining a concentration of deoxypyridinoline in the sample comprises antibodies.

20. The kit according to claim 15, wherein the means for determining a concentration of pyridinoline in the sample comprises antibodies.
